(19) United States
(12) Patent Application Publication
(10) Pub. No.: US 2019/0000876 A1
(43) Pub. Date: Jan. 3, 2019

(54) STABILIZED ANTISEPTIC PREPARATIONS

(71) Applicant: Tecuro AG, Luzern (CH)

(72) Inventors: Otto von Stetten, Aachen (DE); Corinna Hengsberger, Schopfheim (DE); Markus Benjamin Bannwarth, St. Gallen (CH); Luciano Fernandes Boesel, St. Gallen (CH)

(73) Assignee: Tecuro AG, Luzern (CH)

(21) Appl. No.: 15/763,838

(22) PCT Filed: Sep. 20, 2016
(86) PCT No.: PCT/EP2016/072251

§ 371 Date: Sep. 21, 2018

(30) Foreign Application Priority Data
Sep. 29, 2015 (CH) 01417/15

(51) Int. Cl. A61K 33/26; A61K 33/30; A61K 33/34; A61K 47/22; A61K 47/10; A61K 47/36; A61K 47/32; A61K 47/02; A61K 9/00; A61K 9/08; A61P 31/02

(52) U.S. Cl. CPC A61K 33/26; A61K 33/30; A61K 33/34; A61K 47/22; A61K 47/10; A61K 47/36; A61K 31/194; A61K 47/02; A61K 9/0014; A61K 9/08; A61P 31/02; A61K 2300/00; A61K 47/32

(57) ABSTRACT

A stabilized antiseptic preparation comprises at least one vitamin, and/or at least one metal ion, and/or at least one surface-active compound, and/or at least one gellant, and/or at least one antimicrobial agent from the group of quaternary ammonium compounds, and/or at least one other antimicrobial agent selected from among the group: organic and inorganic acids or their salts, esters, amides, aliphatic or aromatic monoaldehydes or dialdehydes, guanidines, pyridines or pyrimidines.

### TECHNICAL FIELD

The invention relates to stabilized antiseptic preparations for topical formulations which can be used in the human and veterinary field for treatments in the wound region, for the microbiological sanitization of skin surfaces, for disinfection of the skin and the hands or for wound and bladder rinses.

### TECHNOLOGICAL BACKGROUND

Very effective antiseptic formulations are known from literature, which, in addition to a metal ion, contain further ingredients but do not have the disadvantages of silver-based antimicrobiological preparations.

DE 19936428 describes a formulation which, in addition to iron(III) ions, contains ascorbic acid and further organic acids.

EP 2522223 discloses combined disinfection and decontamination agents with increased activity which contain at least one vitamin, at least one metal ion, at least one surface-active compound and at least one antimicrobial agent from the group of quaternary ammonium compounds, as well as optionally further antimicrobially active substances. These agents can be used for the decontamination or disinfection of living and non-living surfaces.

EP 1881763 describes decontamination solutions and their use for the denaturation, modification, degradation, solubilisation and removal of proteins, nucleic acid molecules and microorganisms which consist of at least one vitamin, at least one metal ion and at least one surface-active compound. For the latter, uses as a therapeutic agent or disinfection agent are indicated in EP 2170398.

Contrary to the indications in the cited documents, all these formulations are not stable, i.e. they discolour as a solution, and their antimicrobial activity decreases markedly. The solutions from EP 1881763, which contain e.g. copper, change their colour from light blue to brownish and then brown. At the same time, precipitation can be observed.

There is therefore a general need for progresses in this area

### OBJECT OF THE INVENTION

The object of the invention is to provide an antiseptic preparation or composition which does not have the aforementioned and other disadvantages. In particular, such an antiseptic preparation should be stabilized for a certain time in such a way that it retains the activity required for the intended use.

These and other objects are achieved through a preparation according to the invention in accordance with the independent claim. Further preferred embodiments are given in the dependent claims.

### SUMMARY OF THE INVENTION

One aspect of the invention relates to stabilized antiseptic preparations.

Stabilized antiseptic preparations according to the invention comprise at least one vitamin and/or at least one metal ion and/or at least one surface-active compound and/or at least one gellant and/or at least one antimicrobial agent from the group of quaternary ammonium compounds and/or at least one other antimicrobial agent selected from among the group consisting of: organic and inorganic acids or their salts, esters, amides, aliphatic or aromatic monoaldehydes or dialdehydes, guanidines, pyridines or pyrimidines.

A preparation according to the invention advantageously has a pH of the mixture in the range of 0.5 to 8.5.

At least one compound, selected from the group of vitamins A, B, C and E, is advantageously contained as vitamins, their salts or acid derivatives.

Advantageously, the metal ions come from the metals of period 4 and of the subgroups I, II or VIII of the Periodic Table of Elements, particularly advantageously iron, copper and zinc. The metals are advantageously present in the form of their salts with acids or bases, as oxides or organometallic compounds.

In a preparation according to the invention, at least one compound selected from the group of the anionic, non-ionic, amphoteric or cationic surfactants, or suitable mixtures with one another or among each other, is advantageously contained as the surface-active substance.

The gellant is advantageously selected from the group of polysaccharides, preferably agarose, carrageenan, pectin, xanthan, alginic acid and alginates, silicon dioxide, polyvinylpyrrolidones, polyvinyl alcohols, polymethacrylates, preferably poly(hydroxymethacrylates) or poly(N-isopropylmethacrylates) or polyacrylic acids, or suitable mixtures thereof.

In a preparation according to the invention, it is advantageous that the other antimicrobial agent is one or several aliphatic or aromatic organic acids or an inorganic acid or its salt, ester or amide. This other antimicrobial agent is particularly advantageously selected from the group consisting of formic acid, acetic acid, bromoacetic acid, glycolic acid, propionic acid, glyoxylic acid, lactic acid, citric acid, tartaric acid, malonic acid, maleic acid, fumaric acid, pyrrolidonecarboxylic acid, sorbic acid, undecylenic acid, undecynoic acid, benzoic acid, hydroxybenzoic acid, salicylic acid, dehydroacetic acid, 4-hydroxybenzoic acid ester, dimethyl carbonate, chloroacetamide, 2-chloro-N-(hydroxymethyl)acetamide, salicylanilide, phosphoric acid, sulfuric acid, hydrochloric acid, boric acid, sulfurous acid, nitric acid and/or carbonic acid.

In a preparation according to the invention, the vitamin is advantageously contained in amounts of 0.1 mM to 1000 mM.

In a preparation according to the invention, the metal ion is advantageously contained in amounts of 0.01 mM to 100 mM.

In a preparation according to the invention, the surface-active substance is advantageously contained in amounts of 0.01% to 35% by weight of the preparation.

In a preparation according to the invention, the gellant is advantageously contained in amounts of 0.25% to 4.5%, preferably 0.5% to 3.5%, particularly preferably 0.75% to 2.5%, relative to the preparation.

A preparation according to the invention can be advantageously incorporated into a topical, dermatological or cosmetic formulation, e.g. gel, lotion, solution, cream, ointment, powder, microspheres, liposomes, foam, stick, rinse, spray, etc.

A preparation according to the invention can advantageously also be applied to a carrier material.

A further aspect of the invention relates to the use of stabilized antiseptic preparations according to the invention, in particular stabilized antiseptic preparations as mentioned above for use in the treatment of wounds in the human and veterinary field, and/or for the microbiological sanitization of skin surfaces.

In such a use, the preparation is advantageously used in conjunction with a compress or wound dressing, preferably a bandage, a plaster and/or another wound covering.

Also advantageously, in such a use, the preparation is used in foamed form.

Furthermore, in such a use, the preparation can advantageously be used as a wound rinse solution or moisture component of a wound dressing, or as a rinse solution or moisture component for the microbiological sanitization of skin surfaces.

A further advantageous variant of a use according to the invention of a stabilized antiseptic preparation relates to a stabilized antiseptic preparation according to the invention for use for the prevention and/or treatment of pathological conditions in the urogenital region.

The preparation is particularly advantageously used as a bladder rinse.

A yet further advantageous variant of a use according to the invention of a stabilized antiseptic preparation relates to a stabilized antiseptic preparation according to the invention for use for the microbiological sanitization of the urogenital region.

The preparation is particularly advantageously applied in a topical formulation in the urogenital region, preferably in the region between the anus and the urethra, or it is applied to a dressing, e.g. a panty liner, and placed in the region between the anus and the urethra, optionally interlabially.

Yet another advantageous variant of a use according to the invention of a stabilized antiseptic preparation relates to a stabilized antiseptic preparation according to the invention for use in skin and hand disinfection.

### BRIEF DESCRIPTION OF THE DRAWINGS

The mode of action of the disclosed antiseptic compositions is explained below with reference to drawings.

FIG. 1 shows the experimentally determined stabilizing action of agarose as gellant on an antiseptic solution.

FIG. 2 shows the release of the antiseptic agent for various layer thicknesses and gellants.

### DETAILED DESCRIPTION OF THE INVENTION

The following examples are intended to explain the invention without limiting its scope.

Surprisingly, it has been found that antiseptic formulations can be stabilized by adding a gellant, e.g. polysaccharides, alginates, polyvinyl alcohols, etc.

At the same time, it has been found that, with such novel formulations, the release of the active principle of action can be delayed over a longer period of time, so that the effective concentration can be maintained for a longer time at the site of action and the therapeutic purpose can thus be better achieved.

Preparations according to the invention contain at least one synergistic mixture of at least one vitamin, at least one metal ion, at least one surface-active compound and at least one gellant. At least one antimicrobial agent from the group of quaternary ammonium compounds and/or at least one further antimicrobial agent selected from the group consisting of organic and inorganic acids or their salts, esters, amides, aliphatic or aromatic monoaldehydes or dialdehydes, guanidines, pyridines or pyrimidines may optionally be present.

The pH of preparations according to the invention is in the range of 0.5 to 8.5, in particular of 1 to 7, preferably of 2 to 6, particularly preferably of 2 to 4.5. The pH can be adjusted with organic acids such as formic acid, acetic acid, propionic acid, citric acid, tartaric acid, malic acid, inorganic acids such as hydrochloric acid, sulfuric acid, phosphoric acid, etc., or also buffer systems such as citrate, phosphate, tris(hydroxymethyl)aminomethane (Tris), succinate, carbonate, borate, oxalate, glycine, tartrate, acetate buffer, etc., or suitable mixtures thereof. The antiseptic activity is given over the entire indicated pH range.

The vitamins in preparations according to the invention are selected from the group of vitamins A, B, C and E and are present as vitamins, their salts and/or acidic derivatives. Mixtures of several vitamins can be used. Vitamin C, riboflavin and niacin are preferably used. The amounts of vitamins are preferably between 0.1 mM and 1000 mM, in particular between 1 mM and 500 mM, preferably between mM and 300 mM, and very particularly preferably between 1 mM and 100 mM.

The preparations according to the invention contain metal ions of the metals of period 4 and of the subgroups I, II, or VIII of the Periodic Table of Elements. The ions can in this case be in the form of their salts with acids or bases, as oxides or organometallic compounds. Either individual metal ions or mixtures of several ions are used. Ions of the metals iron, cobalt, nickel, copper or zinc are preferably contained in amounts of 0.01 mM to 100 mM, preferably of 0.4 mM to 50 mM, in particular of 1 mM to 30 mM, very particularly preferably of 1 mM to 10 mM.

Surface-active substances are required in the mixture for the synergistic action of the antiseptic preparations. These are selected from the group of anionic, non-ionic, amphoteric or cationic surfactants. These are, e.g. alkyl ether sulfates, alkyl and/or aryl sulfonates, alkyl sulfates, olefin sulfonates, amphoteric surfactants, betaines, alkylamidoalkylamines, alkyl-substituted amino acids and/or imino acids, acylated amino acids, sugar surfactants, quaternary ammonium compounds, etc. The surfactants are used either individually or in suitable mixtures with one another. The typical amounts are between 0.1% by weight and 35% by weight, preferably 0.2% by weight and 30% by weight, particularly preferably 0.5% by weight and 20% by weight, very particularly preferably 0.5% by weight and 15% by weight.

Surprisingly, it has been found that the stability and thus at the same time the antiseptic activity of antiseptic preparations can be significantly improved by the addition of gellants. As gellants are selected polysaccharides, preferably agarose, carrageenan, pectin, xanthan, alginic acid and alginates, silicon dioxide, polyvinylpyrrolidones, polyvinyl alcohols, polymethacrylates, preferably poly(hydroxymethacrylates) or poly(N-isopropylmethacrylates) or polyacrylic acids, or suitable mixtures thereof. Agarose and carrageenan are particularly preferred. The gellants are used in amounts of 0.25% to 4.5%, preferably 0.5% to 3.5%, particularly preferably 0.75% to 2.5%, relative to the preparation.

The following table shows various gellants and their suitability for antiseptic preparations according to the invention.

TABLE

| | | Solubility | |
|---|---|---|---|
| Gellant | Type of gelation | at acidic pH | Suitability |
| Chitosan | pH > 5 | yes | moderate |
| Alginate | Addition of Ca²⁺ | bad | moderate |
| | ions | | |
| Agarose | Temperature | yes | yes |
| | increase and | | |
| | cooling | | |
| Carrageenan | Temperature | yes | yes |
| | increase and | | |
| | cooling | | |
| Pectin | Temperature | yes | yes |
| | increase, | | |
| | addition of sugar | | |
| | and cooling | | |
| Hyaluronic acid | Addition of | bad | moderate |
| | polylysine or | | |
| | other cations | | |
| Poly(vinyl | Photocrosslinking | yes | yes |
| pyrrolidone) | | | |
| Poly(vinvl | Radical | yes | yes |
| alcohol) | polymerisation | | |
| | and crosslinking | | |
| Poly(hydroxymeth | Radical | yes | yes |
| acrylate) | polymerisation | | |
| | and crosslinking | | |
| Poly(N- | Radical | yes | yes |
| hydroxymeth | polymerisation | | |
| acrylate) | and crosslinking | | |
| Poly (acrylic | Radical | yes | yes |
| acid) | polymerisation | | |
| | and crosslinking | | |

Because of poor solubility in the acidic pH range, as is typical for applications in the biodecontamination range, alginate and hyaluronic acid are less suitable. Chitosan in turn cannot be gelled in the acidic pH range <5.

For the preparation of antiseptic preparations according to the invention, it is advantageous to first prepare the gel and then to incorporate the further components of the antiseptic preparation, so that the stabilized antiseptic preparation is finally obtained. The preparations according to the invention can contain further antimicrobial agents, selected from the group of quaternary ammonium compounds, e.g. benzalkonium chloride, procuronium chloride, trimethylundecylammonium chloride, benzethonium chloride, etc.

As other antimicrobial agents can be incorporated aliphatic or aromatic organic acids or inorganic acids or their salts, esters or amides, these antimicrobial agents being selected from the group consisting of formic acid, acetic acid, bromoacetic acid, glycolic acid, propionic acid, glyoxylic acid, lactic acid, citric acid, tartaric acid, malonic acid, maleic acid, fumaric acid, pyrrolidonecarboxylic acid, sorbic acid, undecylenic acid, undecynoic acid, benzoic acid, hydroxybenzoic acid, salicylic acid, dehydroacetic acid, 4-hydroxybenzoic acid ester, dimethyl carbonate, chloroacetamide, 2-chloro-N-(hydroxymethyl)acetamide, salicylanilide, phosphoric acid, sulfuric acid, hydrochloric acid, boric acid, sulfurous acid, nitric acid and/or carbonic acid. Depending on the intended use, it may be advantageous to incorporate the stabilized antiseptic preparations into topically applicable formulations according to the usual methods in the preparation of cosmetics and/or the pharmaceutical-technological methods. In this context, topical means that the formulations or preparations, in the human and veterinary field, are brought into contact with living surfaces on the body, e.g. skin, hair, etc. and/or within the body, e.g. bladder, oral cavity, vagina, etc. These formulations can be, e.g. gel, lotion, solution, cream, ointment, powder, microspheres, liposomes, foam, stick, rinse, spray, etc.

In one embodiment of the invention, either the antiseptic preparations are applied as such and/or the formulations mentioned are applied onto a carrier material. These materials may comprise, e.g. gauze, compresses, wound dressings, plasters, panty liners, etc. On the other hand, the preparations or formulations can be applied directly to the living surface, e.g. skin, wound, etc., and then covered with a compress, wound dressing, bandage, plaster or other wound covering. In order to intensify the contact with the living surface, bio or mucoadhesive substances can be admixed to the preparations and/or formulations.

Preparations or formulations according to the invention can also be used as such for wound treatment. In certain cases, it is advantageous to use the preparations in foamed form, for example in order to take care of wounds which are sensitive to pain.

For the microbiological sanitization of wounds or skin surfaces, preparations and/or formulations according to the invention can be used as wound rinse solution or moisture component of a wound dressing or as rinse solution or moisture component.

Women in particular frequently suffer from recurrent bladder inflammation, which is in most cases caused by E. coli bacteria which migrate from the anus into the urethra and further into the bladder. If the preparations or formulations according to the invention are used in the region between the anus and the urethra, these pathological conditions can be prevented and/or treated. For this purpose, the preparations are applied as such and/or in the form of formulations such as gel, lotion, solution, cream, ointment, powder, microspheres, liposomes, foam, stick, rinse, spray, etc. in this region. Thus, the urogenital region is microbiologically sanitized. In another embodiment of the invention, the preparations or formulations are applied to a panty liner and, as usual, placed in the urogenital region, the panty liner optionally covering the region including the anus and the vulva, or else being placed between the labia.

In a further embodiment, the preparations are used as bladder rinse, these sanitizing the bladder microbiologically.

In the case of skin and hand disinfection, there is the problem that bacteria come to the surface from the deeper layers of the skin shortly after disinfection and thus compromise the antiseptic state. This is the case in particular in surgical hand disinfection when considerable bacterial concentrations partially accumulate in the glove during an operation lasting several hours. Since gloves used today frequently tear or are perforated, a risk to the patient thus occurs. When the preparations and/or formulations according to the invention are used, a sufficient concentration of the antiseptics can be maintained over an extended period of time, since a targeted release over an extended period of time can be achieved through the use of the gellant. The release can thus be controlled, on the one hand, via the gel concentration and, on the other hand, via the layer thickness of the gel.

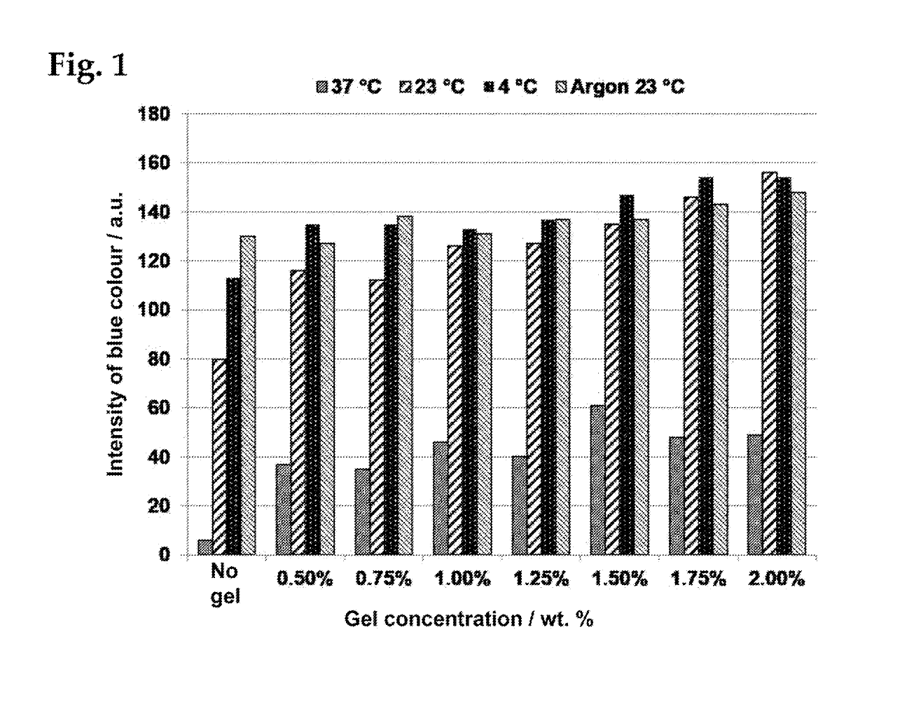

FIG. 1 shows, using the example of agarose, the stabilizing action of various gellant concentrations on an antiseptic solution containing ascorbic acid, citric acid, tartaric acid, sodium lauryl sulfate and copper(II) chloride. Under usual circumstances, the solutions discolour rapidly, i.e. they lose their bluish colour, become brownish and at the same time lose activity. The bluish colour can therefore be regarded as an indicator of stability and activity. For the verification of the stabilization, gels having different gellant concentrations were stored for 18 days at different temperatures. The blue colour fraction was then colorimetrically determined. The reference used was a preparation which was stored at 23° C. under protective gas (argon).

The stabilizing action of the gellants can be clearly seen from the image. In particular, at higher temperatures, for example at body temperature (37° C.), the relative stabilization is considerable. Thus, with a gellant concentration of already 0.5% by weight of agarose, the remaining concentration is six times higher than without stabilization.

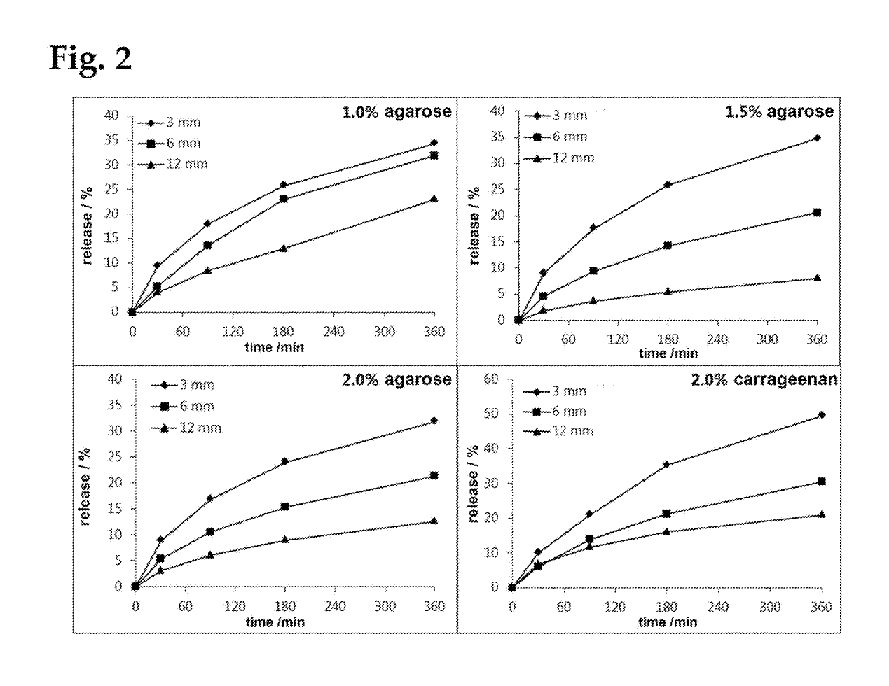

To determine the release of the antiseptic agents, the concentration of copper in the surrounding medium over time was measured as a leading indicator. The release kinetics as shown in FIG. 2 could be determined for various gellants (agarose, carrageenan), various concentrations in % by weight, and various layer thicknesses (3, 6 and 12 mm).

It can be clearly seen that the release can be controlled both via the gel concentration and via the layer thickness. This is important for wound care, microbiological sanitization of the urogenital region and, in particular, skin and hand disinfection. The present invention is not limited in its scope to the specific embodiments described herein. Rather, in addition to the examples disclosed herein, various further modifications of the present invention, which likewise fall within the scope of protection of the claims, are apparent to the person skilled in the art from the description and the associated figures. In addition, various references are cited in the description, the disclosure content of which is hereby incorporated in its entirety into the description for means of reference.

1. A stabilized antiseptic preparation comprising:
at least one vitamin(s), a salt or acid derivative thereof and/or at least one metal ion and/or at least one surface-active compound and/or at least one gellant and/or at least one antimicrobial agent from the group of quaternary ammonium compounds and/or at least one further antimicrobial agent selected from the group consisting of: organic and inorganic acids or their salts, esters, amides, aliphatic or aromatic monoaldehydes or dialdehydes, guanidines, pyridines, pyrimidines and combinations thereof.

2. The stabilized antiseptic preparation according to claim 1, wherein the mixture has a pH in a range of 0.5 to 8.5.

3. The stabilized antiseptic preparation according to claim 1, wherein as the at least one vitamin(s), the salt or acid derivative thereof is selected from the group consisting of vitamins A, B, C, E and combinations thereof.

4. The stabilized antiseptic preparation according to claim 1, wherein the at least one metal ions is/are metal(s) of period 4 and subgroups I, II or VIII of the Periodic Table of Elements, preferably iron, copper and zinc.

5. The stabilized antiseptic preparation according to claim 4, wherein the metal is/are present in the form of their salts with acids or bases, as oxides or organometallic compounds.

6. The stabilized antiseptic preparation according to claim 1, wherein the at least one surface-active substance is selected from the group consisting of the anionic, non-ionic, amphoteric or cationic surfactants, and suitable mixtures thereof.

7. The stabilized antiseptic preparation according to claim 1, wherein the gellant is selected from the group consisting of polysaccharides, preferably agarose, carrageenan, pectin, xanthan, alginic acid and alginates, silicon dioxide, polyvinylpyrrolidones, polyvinyl alcohols, polymethacrylates, preferably poly(hydroxy methacrylates) or poly(N-isopropylmethacrylates) or polyacrylic acids, and suitable mixtures thereof.

8. The stabilized antiseptic preparation according to claim 1, wherein the further antimicrobial agent is an aliphatic or aromatic organic acid or an inorganic acid or its salt, ester or amide, and is selected from the group consisting of formic acid, acetic acid, bromoacetic acid, glycolic acid, propionic acid, glyoxylic acid, lactic acid, citric acid, tartaric acid, malonic acid, maleic acid, fumaric acid, pyrrolidonecarboxylic acid, sorbic acid, undecylenic acid, undecynoic acid, benzoic acid, hydroxybenzoic acid, salicylic acid, dehydroacetic acid, 4-hydroxybenzoic acid ester, dimethyl carbonate, chloroacetamide, 2-chloro-N-(hydroxymethyl)acetamide, salicylanilide, phosphoric acid, sulfuric acid, hydrochloric acid, boric acid, sulfurous acid, nitric acid, carbonic acid and mixtures thereof.

9. The stabilized antiseptic preparation according to claim 1, wherein the at least one vitamin is contained in amounts of 0.1 mM to 1000 mM.

10. The stabilized antiseptic preparation according to claim 1, wherein the at least one metal ion is present in amounts of 0.01 mM to 100 mM.

11. The stabilized antiseptic preparation according to claim 1, wherein the surface-active substance is contained in amounts of 0.01% to 35% by weight of the preparation.

12. The stabilized antiseptic preparation according to claim 1, wherein the gellant is contained in amounts of 0.25% to 4.5%, preferably 0.5% to 3.5%, particularly preferably 0.75% to 2.5%, relative to the preparation.

13. The stabilized antiseptic preparation according to claim 1, wherein it is incorporated into a topical, dermatological or cosmetic formulation, e.g. gel, lotion, solution, cream, ointment, powder, microspheres, liposomes, foam, stick, rinse, spray, etc.

14. The stabilized antiseptic preparation according to claim 1, wherein it is applied to a carrier material.

15. A method for treating a wound in a human and veterinary field and/or in a microbiological sanitization of a skin surfaces comprising:
providing the stabilized antiseptic preparation according to claim 1 and administering the preparation in a treating of wounds and/or in the microbiological sanitization of skin surface effective amount to the wound or skin surface.

16. The method according to claim 15, wherein the preparation is used in conjunction with a compress or wound dressing, preferably a bandage, a plaster and/or another wound covering.

17. The method according to claim 15, wherein the preparation is used in foamed form.

18. The method according to claim 15, wherein the preparation is used as a wound rinse solution or moisture component of a wound dressing, or as a rinse solution or moisture component for the microbiological sanitization of skin surfaces.

19. A method for prevention and/or treatment of pathological conditions in an urogenital region comprising:
providing the stabilized antiseptic preparation according to claim 1 and administering the preparation in a preventing and/or treating of pathological condition in the urogenital region effective amount to the urogenital region.

20. The method according to claim 19, wherein the preparation is used as a bladder rinse.

21. The method of claim 19, wherein the urogenital region is microbiologically sanitized.

22. The method according to claim 21, wherein the preparation is applied in the urogenital region, preferably in the region between the anus and the urethra, in a topical formulation.

23. The method according to claim 21, wherein the preparation is applied onto a dressing, e.g. a panty liner and placed in the region between the anus and the urethra, optionally interlabially.

24. The stabilized antiseptic preparation according to claim 15, wherein the microbiological sanitization is skin and hand disinfection.
